(19) United States
(12) Patent Application Publication
(10) Pub. No.: US 2007/0258967 A1
(43) Pub. Date: Nov. 8, 2007

(54) PURIFICATION METHOD OF REDUCED COENZYME Q10

(75) Inventors: Takahiro Ueda, Kobe-shi (JP); Tadao Ono, Kobe-shi (JP); Shiro Kitamura, Akashi-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Kaneka Corporation, Osaka-shi (JP)

(21) Appl. No.: 11/741,522

(22) Filed: Apr. 27, 2007

(30) Foreign Application Priority Data
Apr. 28, 2006 (JP) 2006-126900

(51) Int. Cl. A61K 38/43; C07C 39/10

(52) U.S. Cl. 424/94.1; 568/766

(57) ABSTRACT

The present invention provides reduced coenzyme Q₁₀ with a low reduced coenzyme Q₁₀ analog content, which is useful as a food, nutritional product, nutritional supplement, animal drug, drink, feed, cosmetic, pharmaceutical product, therapeutic drug, prophylactic drug and the like, and a production method of the reduced coenzyme Q₁₀. The present invention also provides a method of producing a reduced coenzyme Q₁₀ crystal or reduced coenzyme Q₁₀-containing composition, which includes subjecting reduced coenzyme Q₁₀ to chromatography under oxidation preventive conditions, or purifying oxidized coenzyme Q₁₀ by chromatography and converting the oxidized coenzyme Q₁₀ to reduced coenzyme Q₁₀. According to this method, high quality reduced coenzyme Q₁₀ containing not more than 1 wt %, relative to reduced coenzyme Q₁₀, of at least one reduced coenzyme Q₁₀ analog selected from the group consisting of cis-reduced coenzyme Q₁₀, reduced coenzyme Q₁₁ and ubichromenol, can be obtained.

### BACKGROUND OF THE INVENTION

Coenzyme Q₁₀ is locally present in mitochondria, lysosome, Golgi body, microsome, peroxisome, cell membrane and the like and is a substance indispensable to the functional maintenance of the body. It is known to be involved in the activation of ATP production as a constituent component of the electron transport system in mitochondria, antioxidant action in the body and membrane stabilization, and is used for food, pharmaceutical agent, cosmetic and the like. The coenzyme Q₁₀ includes oxidized coenzyme Q₁₀ and reduced coenzyme Q₁₀. It is known that reduced coenzyme Q₁₀ can be obtained, for example, by purifying coenzyme Q₁₀ obtained according to a conventionally-known method such as synthesis, fermentation, extraction from a naturally occurring substance and the like (see JP-A-10-109933). JP-A-10-109933 describes that, in this case, oxidized coenzyme Q₁₀ contained in the above-mentioned reduced coenzyme Q₁₀ can be reduced with a general reducing agent such as sodium borohydride, sodium dithionite (sodium hydrosulfite) and the like, and then purified, and that the reduced coenzyme Q₁₀ can also be obtained by a method comprising reacting existing highly pure coenzyme Q₁₀ with the above-mentioned reducing agent.

When reduced coenzyme Q₁₀ is obtained by the aforementioned synthesis, fermentation, extraction from a naturally occurring substance and the like, various reduced coenzyme Q₁₀ analogs (e.g., cis-reduced coenzyme Q₁₀, ubichromenol, reduced coenzyme Q₁₁ etc.) are contained therein. In general, a method including crystallization of the object product from the solution (crystal precipitation method) is effective for the removal of impurity and a crystallization method of the aforementioned reduced coenzyme Q₁₀ analog is already known (see WO 03/06408 and WO 03/06409).

### SUMMARY OF THE INVENTION

According to the investigation of the present inventors, it was clarified that reduced coenzyme Q₁₀ analogs such as cis-reduced coenzyme Q₁₀, reduced coenzyme Q₁₁, and ubichromenol could not be easily removed by conventional methods such as crystal precipitation method and the like.

In view of the above-mentioned situation, the present invention aims at provision of a production method of reduced coenzyme Q₁₀, which is capable of conveniently removing the above-mentioned analogs, and high quality reduced coenzyme Q₁₀ having a low content of the analogs.

The present inventors have conducted intensive studies in an attempt to solve the above-mentioned problems and found that analogs of reduced coenzyme Q₁₀, which are generally difficult to remove, can be efficiently removed under particular chromatography conditions, and a substantially pure reduced coenzyme Q₁₀ having a low content of a cis-reduced coenzyme Q₁₀, reduced coenzyme Q₁₁, and ubichromenol and the like can be obtained, which resulted in the completion of the present invention.

Accordingly, the present invention provides the following embodiments.
[1] A substantially pure reduced coenzyme Q₁₀ crystal wherein the content of at least one reduced coenzyme Q₁₀ analog selected from the group consisting of cis-reduced coenzyme Q₁₀, reduced coenzyme Q₁₁, and ubichromenol is not more than 1 wt %.
[2] The reduced coenzyme Q₁₀ crystal of [1], wherein the crystal comprises not more than 1 wt % of cis-reduced coenzyme Q₁₀.
[3] The reduced coenzyme Q₁₀ crystal of [1], wherein the crystal comprises not more than 1 wt % of reduced coenzyme Q₁₁.
[4] The reduced coenzyme Q₁₀ crystal of [1], wherein the crystal comprises not more than 1 wt % of ubichromenol.
[5] The reduced coenzyme Q₁₀ crystal of [1]-[4], wherein the crystal comprises not more than 3 wt % of cis-reduced coenzyme Q₁₀, reduced coenzyme Q₁₁, and ubichromenol.
[6] The reduced coenzyme Q₁₀ crystal of [1], wherein the crystal comprises not more than 1 wt % of each of cis-reduced coenzyme Q₁₀, reduced coenzyme Q₁₁, and ubichromenol.
[7] A reduced coenzyme Q₁₀-containing composition comprising reduced coenzyme Q₁₀, and not more than 1 wt %, relative the reduced coenzyme Q10, of a reduced coenzyme Q₁₀ analog selected from the group consisting of cis-reduced coenzyme Q₁₀, reduced coenzyme Q₁₁, and ubichromenol.
[8] The reduced coenzyme Q₁₀-containing composition of [7], wherein the composition comprises not more than 1 wt % of cis-reduced coenzyme Q₁₀ relative to the reduced coenzyme Q₁₀.

[9] The reduced coenzyme Q₁₀-containing composition of [7], wherein the composition comprises not more than 1 wt % of reduced coenzyme Q₁₁ relative to the reduced coenzyme Q₁₀.
[10] The reduced coenzyme Q₁₀-containing composition of [7], wherein the composition comprises not more than 1 wt % of ubichromenol relative to the reduced coenzyme Q₁₀.
[11] The reduced coenzyme Q₁₀-containing composition of [7], wherein the composition comprises not more than 3 wt % of cis-reduced coenzyme Q₁₀, reduced coenzyme Q₁₁, and ubichromenol relative to the reduced coenzyme Q₁₀.
[12] The reduced coenzyme Q₁₀ crystal of claim [7], wherein the composition comprises not more than 1 wt % of each of cis-reduced coenzyme Q₁₀, reduced coenzyme Q₁₁, and ubichromenol relative to the reduced coenzyme Q₁₀.
[13] A method of producing the reduced coenzyme Q₁₀ crystal or reduced coenzyme Q₁₀-containing composition, which comprises

providing a solution comprising reduced coenzyme Q₁₀, at least one reduced coenzyme Q₁₀ analog selected from the group consisting of cis-reduced coenzyme Q₁₀, reduced coenzyme Q₁₁, and ubichromenol, and a solvent, and

subjecting the solution to chromatography to reduce the concentration of the at least one reduced coenzyme Q₁₀ analog to not more than 1 wt % relative to the reduced coenzyme Q₁₀.
[14] The method of [13], wherein the chromatography is column chromatography.
[15] The method of [13], wherein a carrier of the chromatography is silica gel or chemically-modified silica gel.
[16] The method of [15], wherein the chemically-modified silica gel is silica gel modified by an octadecyl group.
[17]. The method of [13], wherein the chromatography is performed under an environment where reduced coenzyme Q₁₀ is protected from oxidation.
[18] The method of [13], wherein subjecting the solution to chromatography comprises contacting a carrier with the solution and then contacting the carrier with a developing solvent.
[19] The method of [18], wherein the developing solvent is a mixed solvent of hexane, 2-propanol, and acetic acid and the reduced coenzyme Q₁₀ analog is cis-reduced coenzyme Q₁₀.
[20] The method of [18], wherein the developing solvent is selected from the group consisting of hexane, methanol, ethanol and mixed solvents thereof, and the reduced coenzyme Q₁₀ analog is one or both of reduced coenzyme Q₁₁ and ubichromenol.
[21] A method of producing a reduced coenzyme Q₁₀ crystal or reduced coenzyme Q₁₀-containing composition, which comprises

providing a solution comprising (a) oxidized coenzyme Q₁₀, (b) at least one impurity selected from the group consisting of cis-oxidized coenzyme Q₁₀, oxidized coenzyme Q₁₁, and ubichromenol, and (c) a solvent,

subjecting the solution to chromatography to reduce the content of the at least one impurity, thereby producing a purified oxidized coenzyme Q₁₀, and

converting the purified oxidized coenzyme Q₁₀ to a reduced coenzyme Q₁₀ that contains not more than 1 wt % relative to the reduced coenzyme Q₁₀ of (a) cis-reduced coenzyme Q₁₀ if the impurity is cis-oxidized coenzyme Q₁₀, (b) reduced coenzyme Q₁₁ if the impurity is oxidized coenzyme Q₁₁, or (c) ubichromenol if the impurity is ubichromenol.
[22] The method of [21], wherein subjecting the solution to chromatography comprises contacting a carrier with the solution and then contacting the carrier with a developing solvent.
[23] The method of [22], wherein the developing solvent is a mixed solvent comprising at least two of the group consisting of hexane, 2-propanol, and ethyl acetate, and the impurity is cis-oxidized coenzyme Q₁₀.
[24] The method of [22], wherein the developing solvent is selected from the group consisting of hexane, methanol, ethanol and mixed solvents thereof, and the impurity is one or both of oxidized coenzyme Q₁₁ and ubichromenol.

### DETAILED DESCRIPTION OF THE INVENTION

According to the present invention, analogs of reduced coenzyme Q₁₀, which are generally difficult to remove, can be removed conveniently, and high quality reduced coenzyme Q₁₀ can be economically obtained with good workability.

Particularly, when reduced coenzyme Q₁₀ is added to food, pharmaceutical products, and the like, a decrease in the absolute amount of reduced coenzyme Q₁₀, which is the active ingredient, can be suppressed by decreasing the content of reduced coenzyme Q₁₀ analogs such as cis-reduced coenzyme Q₁₀, reduced coenzyme Q₁₁, ubichromenol and the like. Moreover, a decrease in the content of such reduced coenzyme Q₁₀ analogs can reduce unpredictable effect caused by ingestion of a foreign substance inherently absent in the human body. While a long-term intake of a large amount of reduced coenzyme Q₁₀ as a food and the like has not been known conventionally, a decrease in the aforementioned adverse effects of impurities is particularly useful in such a situation.

The present invention is explained in detail below. In the present specification, the phrase “coenzyme Q₁₀,” when simply expressed includes the oxidized form, the reduced form, and/or a mixture thereof when they are both present. When trans or cis is not indicated, all-trans type coenzyme Q₁₀ is intended.

The reduced coenzyme Q₁₀ to be used in the present invention can be obtained, for example, by a conventionally known method such as synthesis, fermentation, extraction from a naturally occurring substance and the like. Preferably, the reduced coenzyme Q₁₀ is obtained by synthesis, fermentation, extraction from a naturally occurring substance and the like, or obtained by reduction of oxidized coenzyme Q₁₀, more preferably, obtained by reduction of oxidized coenzyme Q₁₀ with a reducing agent such as ascorbic acids, an ester thereof or a salt thereof, sodium hydrosulfite and the like.

The reduced coenzyme Q₁₀ crystal of the present invention (sometimes to be referred to as the crystal of the present invention) is a substantially pure reduced coenzyme Q₁₀ crystal. A substantially pure reduced coenzyme Q₁₀ crystal refers to a reduced coenzyme crystal that is substantially free of impurities. The crystal preferably comprises less than 10 wt % (e.g., less than 9 wt %, less than 8 wt %, less than 7 wt %, less than 6 wt %, less than 5 wt %, less than 4 wt %, less than 3 wt %, less than 2 wt %, or less than 1 wt %) total impurities (i.e., any substance other than reduced coenzyme Q₁₀). In particular, the crystal has less than 1 wt % of at least one kind of reduced coenzyme Q₁₀ analogs selected from the group consisting of cis-reduced coenzyme Q₁₀, reduced coenzyme Q₁₁ and ubichromenol is not more than 1 wt %.

In the crystal of the present invention, the content of cis-reduced coenzyme Q₁₀ is generally not more than 1 wt % (e.g., not more than 0.9 wt %, not more than 0.8 wt %, not more than 0.7 wt %, not more than 0.6 wt %, not more than 0.5 wt %, not more than 0.4 wt %, not more than 0.3 wt %, not more than 0.2 wt %, and not more than 0.1 wt %), preferably not more than 0.8 wt %, more preferably not more than 0.5 wt %.

In particular, since cis-reduced coenzyme Q₁₀ may not fully exhibit its antioxidant activity due to the steric structure thereof and may act as a pro-oxidant, the risk of developing an oxidation stress can be obliterated by eliminating cis-reduced coenzyme Q₁₀.

In the crystal of the present invention, the content of reduced coenzyme Q₁₁ is generally not more than 1 wt % (e.g., not more than 0.9 wt %, not more than 0.8 wt %, not more than 0.7 wt %, not more than 0.6 wt %, not more than 0.5 wt %, not more than 0.4 wt %, not more than 0.3 wt %, not more than 0.2 wt %, and not more than 0.1 wt %), preferably not more than 0.8 wt %, more preferably not more than 0.5 wt %.

In the crystal of the present invention, the content of ubichromenol is generally not more than 1 wt % (e.g., not more than 0.9 wt %, not more than 0.8 wt %, not more than 0.7 wt %, not more than 0.6 wt %, not more than 0.5 wt %, not more than 0.4 wt %, not more than 0.3 wt %, not more than 0.2 wt %, and not more than 0.1 wt %), preferably not more than 0.8 wt %, more preferably not more than 0.5 wt %.

In the crystal of the present invention, the total content of cis-reduced coenzyme Q₁₀, reduced coenzyme Q₁₁ and ubichromenol is generally not more than 3 wt %, preferably not more than 2 wt %, more preferably not more than 1 wt % (e.g., not more than 0.9 wt %, not more than 0.8 wt %, not more than 0.7 wt %, not more than 0.6 wt %, not more than 0.5 wt %, not more than 0.4 wt %, not more than 0.3 wt %, not more than 0.2 wt %, not more than 0.1 wt %). When the content of the above-mentioned analog is within the above-mentioned range, for example, a decrease in the absolute amount of reduced coenzyme Q₁₀, which is the active ingredient, can be suppressed and an unpredictable influence caused by ingestion of a foreign substance inherently absent in the human body can be reduced and preferably eliminated. Therefore, when the content of the above-mentioned analog is within the above-mentioned range, for example, a long-term, safe use can be possible even when the crystal of the present invention is consumed in a large amount every day in food, supplements, and the like.

In addition, using high quality reduced coenzyme Q₁₀, or a crystal thereof, obtained by the production method described herein as an active ingredient, a high quality reduced coenzyme Q₁₀-containing composition with a low content of reduced coenzyme Q₁₀ analog can be obtained.

That is, the reduced coenzyme Q₁₀-containing composition of the present invention (to be also referred to as the composition of the present invention) includes reduced coenzyme Q₁₀ (crystal) wherein the content of at least one kind of reduced coenzyme Q₁₀ analog selected from the group consisting of cis-reduced coenzyme Q₁₀, reduced coenzyme Q₁₁, and ubichromenol is not more than 1 wt %. In particular, the reduced coenzyme Q₁₀-containing composition comprises reduced coenzyme Q₁₀ and not more than 1 wt %, relative to the reduced coenzyme Q₁₀ (i.e., based on the content of the reduced coenzyme Q₁₀ crystal), of at least one of the reduced coenzyme Q₁₀ analogs selected from the group consisting of cis-reduced coenzyme Q₁₀, reduced coenzyme Q₁₁, and ubichromenol.

The range of cis-reduced coenzyme Q₁₀, reduced coenzyme Q₁₁, and ubichromenol is the same as that mentioned for the above-mentioned crystal of the present invention. When the composition of the present invention is similarly consumed in a large amount every day, for example, in a supplement, a long-term, safe administration can be possible since a decrease in the absolute amount of reduced coenzyme Q₁₀, which is the active ingredient, can be suppressed and an unpredictable influence caused by ingestion of a foreign substance inherently absent in the human body can be eliminated.

While the composition of the present invention can be used as it is, the composition can be processed into a dosage form for oral administration such as a capsule (e.g., a microcapsule, hard capsule, or soft capsule), tablet, syrup, drink and the like.

In addition, the composition can be processed into a dosage form for parenteral administration such as a cream, suppository, toothpaste and the like.

Particularly preferred is a capsule, especially soft capsule.

The capsule base material is not particularly limited, and gelatin derived from beef bones, cattle skin, pig skin, fish skin and the like, and other base materials (e.g., gum stabilizers that can be used as food additives, such as seaweed-derived products (e.g., carageenan, alginic acid and the like), vegetable seed-derived products (e.g., locust bean gum, guar gum and the like), and agents for production (e.g., celluloses) and the like) can also be used.

When processing the reduced coenzyme Q₁₀-containing composition of the present invention into the above-mentioned oral administration form or other form, other materials can be appropriately added depending on the object thereof. The composition can contain a carrier, such as a pharmaceuctically acceptable carrier. The above-mentioned other materials are not particularly limited and, for example, excipient, disintegrant, lubricant, binder, antioxidant, coloring agent, anticoagulant, absorption promoter, solubilizing agent of the active ingredient, stabilizer, and combinations thereof and the like can be used. An active ingredient other than coenzyme Q₁₀ can also be co-present.

The above-mentioned excipient is not particularly limited. For example, sucrose, lactose, glucose, cornstarch, mannitol, crystalline cellulose, calcium phosphate, calcium sulfate and the like can be used as an excipient.

The above-mentioned disintegrant is not particularly limited. For example, starch, agar, calcium citrate, calcium carbonate, sodium hydrogencarbonate, dextrin, crystalline cellulose, carboxymethylcellulose, tragacanth and the like can be used as a disintegrant.

The above-mentioned lubricant is not particularly limited. For example, talc, magnesium stearate, polyethylene glycol, silica, hydrogenated vegetable oil and the like can be used as a lubricant.

The above-mentioned binder is not particularly limited. For example, ethylcellulose, methylcellulose, hydroxypropylmethylcellulose, tragacanth, shellaci gelatin, gum arabic, polyvinylpyrrolidone, polyvinyl alcohol, polyacrylic acid, polymethacrylic acid, sorbitol and the like can be used as a binder.

The above-mentioned antioxidant is not particularly limited. For example, ascorbic acid, tocopherol, vitamin A, β-carotene, sodium hydrogensulfite, sodium thiosulfate, sodium pyrrosulfite, citric acid and the like can be used as an antioxidant.

The above-mentioned coloring agent is not particularly limited. For example, those allowed to be added to pharmaceutical products and food and the like can be used as a coloring agent.

The above-mentioned anticoagulant is not particularly limited. For example, stearic acid, talc, light anhydrous silicic acid, water-containing silicon dioxide and the like can be used as an anticoagulant.

The above-mentioned absorption promoter is not particularly limited. For example, higher alcohols, higher fatty acids, sucrose fatty acid ester, surfactants such as sorbitan fatty acid ester, sorbitan polyoxyethylene fatty acid ester and the like, and the like can be used as an absorption promoter.

The solubilizing agent for the above-mentioned active ingredient is not particularly limited. For example, organic acids such as fumaric acid, succinic acid, malic acid and the like, and the like can be used as a solubilizing agent.

The above-mentioned stabilizer is not particularly limited. For example, benzoic acid, sodium benzoate, ethyl parahydroxybenzoate and the like can be used as a stabilizer.

The active ingredient other than the above-mentioned coenzyme Q₁₀ can be any other suitable active agent, such as, for example, an amino acid, vitamin, mineral, polyphenol, organic acid, saccharides, peptide, protein and the like.

While the amount of the reduced coenzyme Q₁₀ contained in the composition of the present invention is not particularly limited, the weight of the reduced coenzyme Q₁₀ contained in the whole composition is generally not less than about 0.01 wt %, preferably not less than about 0.1 wt %, more preferably not less than about 1 wt %, particularly preferably not less than about 2 wt %, and more preferably not less than about 3 wt %.

While the upper limit is not particularly limited, it is generally not more than about 70%, preferably not more than about 60 wt %, and more preferably not more than about 50 wt % in consideration of the viscosity of the composition and the like.

The production method of the crystal and composition of the present invention characteristically include a step of removing impurities by chromatography. As the impurities that can be removed by chromatography, cis-reduced coenzyme Q₁₀, reduced coenzyme Q₁₁, ubichromenol and the like can be mentioned. Impurities other than the above-mentioned can also be removed by chromatography.

In another embodiment, the production method of the present invention is a production method of reduced coenzyme Q₁₀, which comprises a step of purifying reduced coenzyme Q₁₀ containing at least one reduced coenzyme Q₁₀ analog selected from the group consisting of cis-reduced coenzyme Q₁₀, reduced coenzyme Q₁₁, or ubichromenol by chromatography.

The chromatography is not particularly limited. For example, column chromatography, thin layer chromatography and the like can be used, with preference given to column chromatography.

The carrier to be used for chromatography is not particularly limited. For example, commercially available silica gel and the like can be used. The silica gel to be used as the carrier can be chemically modified. Examples of the silica gel include silica gel chemically modified with an octadecyl group, silica gel chemically modified with an octyl group, silica gel chemically modified with a butyl group, silica gel chemically modified with a trimethyl group, silica gel chemically modified with a phenyl group and the like. Preferred is silica gel chemically modified with an octadecyl group.

Reduced coenzyme Q₁₀ is easily oxidized upon contact with silica gel in the air. Thus, chromatography is desirably performed under an environment where reduced coenzyme Q₁₀ is protected from oxidation.

In one example, oxygen contained in a developing solvent is removed and chromatography is performed under a deoxygenation atmosphere.

Removal of oxygen from a developing solvent and deoxygenation atmosphere can be achieved by substitution with inert gas, reduced pressure, boiling and combination of these. Use of at least the substitution with an inert gas, i.e., inert gas atmosphere, is preferable. Examples of the above-mentioned inert gas include nitrogen gas, helium gas, argon gas, hydrogen gas, carbon dioxide gas and the like, with preference given to nitrogen gas.

It is also possible to prevent oxidation of reduced coenzyme Q₁₀ by the co-presence of a developing solvent and a strong acid having a pKa of 2.5 or below, citric acids, ascorbic acids and the like. It is more preferable to combine the above-mentioned deoxygenation atmosphere and the above-mentioned co-presence of acids.

The developing solvent to be used for chromatography is not particularly limited, and only needs to be selected according to the reduced coenzyme Q₁₀ analog to be removed.

Examples of such a developing solvent include hydrocarbons, fatty acid esters, nitriles, ethers, ketones, alcohols, fatty acids and a mixed solvent thereof and the like.

The hydrocarbons are not particularly limited. For example, aliphatic hydrocarbon, aromatic hydrocarbon, halogenated hydrocarbon and the like can be used. Particularly, aliphatic hydrocarbon and aromatic hydrocarbon are preferable, and aliphatic hydrocarbon is especially preferable.

Aliphatic hydrocarbons can be cyclic or non-cyclic, saturated or unsaturated, and are not particularly limited. Generally, aliphatic hydrocarbons having 3 to 20 carbon atoms, preferably 5 to 12 carbon atoms, can be used. Specific examples include propane, butane, isobutane, pentane, 2-methylbutane, cyclopentane, 2-pentene, hexane, 2-methylpentane, 2,2-dimethylbutane, 2,3-dimethylbutane, methylcyclopentane, cyclohexane, 1-hexene, cyclohexene, heptane, 2-methylhexane, 3-methylhexane, 2,3-dimethylpentane, 2,4-dimethylpentane, methylcyclohexane, 1-heptene, octane, 2,2,3-trimethylpentane, isooctane, ethylcyclohexane, 1-octene, nonane, 2,2,5-trimethylhexane, 1-nonene, decane, 1-decene, p-menthane, undecane, dodecane and the like.

Of these, pentane, 2-methylbutane, cyclopentane, hexane, 2-methylpentane, 2,2-dimethylbutane, 2,3-dimethylbutane, methylcyclopentane, cyclohexane, heptane, 2-methylhexane, 3-methylhexane, 2,3-dimethylpentane, 2,4-dimethylpentane, methylcyclohexane, octane, 2,2,3-trimethylpentane, isooctane and ethylcyclohexane are preferably used.

While aromatic hydrocarbons are not particularly limited, normally, an aromatic hydrocarbon having 6 to 20 carbon atoms, particularly 6 to 12 carbon atoms, especially 7 to 10 carbon atoms, is preferably used. Specific examples include benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene, styrene and the like. It is preferably toluene, xylene, o-xylene, m-xylene, p-xylene, ethylbenzene, cumene, mesitylene, tetralin, butylbenzene, p-cymene, cyclohexylbenzene, diethylbenzene or pentylbenzene, more preferably, toluene, xylene, o-xylene, m-xylene, p-xylene, cumene or tetralin, and most preferably cumene.

Halogenated hydrocarbon can be cyclic or non-cyclic, saturated or unsaturated, and is not particularly limited. In general, a non-cyclic halogenated hydrocarbon is preferably used. A halogenated hydrocarbon having 1 to 6 carbon atoms, particularly 1 to 4 carbon atoms, especially 1 or 2 carbon atoms, is preferably used. Chlorinated hydrocarbon and fluorinated hydrocarbon are preferable, and chlorinated hydrocarbon is particularly preferable. Specific examples include dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1-dichloroethylene, 1,2-dichloroethylene, trichloroethylene, tetrachloroethylene, 1,2-dichloropropane, 1,2,3-trichloropropane, chlorobenzene, 1,1,1,2-tetrafluoroethane and the like. It is preferably dichloromethane, chloroform, carbon tetrachloride, 1,1-dichloroethane, 1,2-dichloroethane, 1,1,1-trichloroethane, 1,1,2-trichloroethane, 1,1-dichloroethylene, 1,2-dichloroethylene, trichloroethylene, chlorobenzene or 1,1,1,2-tetrafluoroethane, more preferably dichloromethane, chloroform, 1,2-dichloroethylene, trichloroethylene, chlorobenzene or 1,1,1,2-tetrafluoroethane.

Fatty acid esters are not particularly limited. For example, propionic acid ester, acetic acid ester, formic acid ester and the like can be used. Particularly, acetic acid ester and formic acid ester are preferable, and acetic acid ester is especially preferable.

While the ester group is not particularly limited, in general, alkyl ester or aralkyl ester having 1 to 8 carbon atoms, preferably alkyl ester having 1 to 6 carbon atoms, more preferably alkyl ester having 1 to 4 carbon atoms, is preferably used.

Examples of propionic acid ester include methyl propionate, ethyl propionate, butyl propionate and isopentyl propionate.

Examples of acetic acid ester include methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate, benzyl acetate and the like. It is preferably methyl acetate, ethyl acetate, propyl acetate, isopropyl acetate, butyl acetate, isobutyl acetate, sec-butyl acetate, pentyl acetate, isopentyl acetate, sec-hexyl acetate or cyclohexyl acetate, more preferably methyl acetate, ethyl acetate, propyl acetate, isopropyl acetate, butyl acetate or isobutyl acetate, and most preferably ethyl acetate.

Examples of formic acid ester include methyl formate, ethyl formate, propyl formate, isopropyl formate, butyl formate, isobutyl formate, sec-butyl formate, pentyl formate and the like. It is preferably methyl formate, ethyl formate, propyl formate, butyl formate, isobutyl formate or pentyl formate, and most preferably ethyl formate.

Nitriles can be cyclic or non-cyclic, saturated or unsaturated, and are not particularly limited. In general, saturated one is used. A nitrile having 2 to 20 carbon atoms, particularly 2 to 12 carbon atoms, especially 2 to 8 carbon atoms, is preferably used. Specific examples include acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, cyanomethyl acetate, cyanoethyl acetate, tolunitrile, benzonitrile, chlorobenzonitrile, bromobenzonitrile, cyanobenzoic acid, nitrobenzonitrile, anisonitrile, phthalonitrile, bromotolunitrile, methylcyanobenzoate, methoxybenzonitrile, acetylbenzonitrile, naphtonitrile, biphenylcarbonitrile, phenylpropionitrile, phenylbutyronitrile, methylphenylacetonitrile, diphenylacetonitrile, naphthylacetonitrile, nitrophenylacetonitrile, chlorobenzyl cyanide, cyclopropanecarbonitrile, cyclohexanecarbonitrile, cycloheptanecarbonitrile, phenylcyclohexanecarbonitrile, tolylcyclohexanecarbonitrile and the like. The nitrile is preferably acetonitrile, propionitrile, succinonitrile, butyronitrile, isobutyronitrile, valeronitrile, cyanomethyl acetate, cyanoethyl acetate, benzonitrile, tolunitrile or chloropropionitrile, more preferably acetonitrile, propionitrile, butyronitrile or isobutyronitrile, and most preferably acetonitrile.

Ethers are not particularly limited and can be cyclic or non-cyclic, saturated or unsaturated. In general, saturated ethers are preferably used.

Ether having 3 to 20 carbon atoms, particularly ether having 4 to 12 carbon atoms, especially ether having 4 to 8 carbon atoms, is preferably used. Specific examples include diethyl ether, methyl tert-butyl ether, dipropyl ether, diisopropyl ether, dibutyl ether, dihexyl ether, ethylvinyl ether, butylvinyl ether, anisole, phenetol, butylphenyl ether, methoxytoluene, dioxane, furan, 2-methylfuran, tetrahydrofuran, tetrahydropyran, ethylene glycol dimethyl ether, ethylene glycol diethyl ether, ethylene glycol dibutyl ether, ethylene glycol monomethyl ether, ethylene glycol monoethyl ether, ethylene glycol dibutyl ether and the like. It is preferably diethyl ether, methyl tert-butyl ether, dipropyl ether, diisopropyl ether, dibutyl ether, dihexyl ether, anisole, phenetol, butylphenyl ether, methoxytoluene, dioxane, 2-methylfuran, tetrahydrofuran, tetrahydropyran, ethylene glycol dimethyl ether, ethylene glycol diethyl ether, ethylene glycol dibutyl ether, ethylene glycol monomethyl ether or ethylene glycol monoethyl ether, more preferably diethyl ether, methyl tert-butyl ether, anisole, dioxane, tetrahydrofuran, ethylene glycol monomethyl ether or ethylene glycol monoethyl ether, more preferably diethyl ether, methyl tert-butyl ether, anisole and the like, and most preferably methyl tert-butyl ether.

Ketones are not particularly limited, and ketone having 3 to 6 carbon atoms is generally preferable. Specific examples include acetone, methylethylketone, methylbutylketone, methylisobutylketone and the like, and particularly, acetone and methylethylketone are preferable, and acetone is particularly preferable.

Alcohols can be cyclic or non-cyclic, saturated or unsaturated, and is not particularly limited. In general, a saturated alcohol is preferably used.

Normally, monovalent alcohol having 1 to 20 carbon atoms, particularly 1 to 12 carbon atoms, especially 1 to 6 carbon atoms, particularly 1 to 5 carbon atoms, particularly 1 to 3 carbon atoms, especially 2 or 3 carbon atoms, divalent alcohol having 2 to 5 carbon atoms or trivalent alcohol having 3 carbon atoms is preferably used.

Examples of a monovalent alcohol include methanol, ethanol, 1-propanol, 2-propanol, 1-butanol, 2-butanol, isobutylalcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol and the like.

The monovalent alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol or cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol or neopentyl alcohol, more preferably methanol, ethanol, 1-propanol, 2-propanol, 1-butanol, 2-butanol, isobutyl alcohol, 2-methyl-1-butanol or isopentyl alcohol, particularly preferably methanol, ethanol, 1-propanol or 2-propanol, further preferably ethanol, 1-propanol or 2-propanol, and most preferably ethanol.

Examples of a divalent alcohol include 1,2-ethanediol, 1,2-propanediol, 1,3-propanediol, 1,2-butanediol, 1,3-butanediol, 1,4-butanediol, 2,3-butanediol, 1,5-pentanediol and the like. The divalent alcohol is preferably 1,2-ethanediol, 1,2-propanediol or 1,3-propanediol, and most preferably 1,2-ethanediol. As a trivalent alcohol, glycerol and the like can be preferably used.

Examples of fatty acids include formic acid, acetic acid, propionic acid and the like. Formic acid and acetic acid are preferable, and acetic acid is more preferable.

Of the above-mentioned solvents, use of a mixed solvent comprising at least two selected from the group consisting of hydrocarbons, alcohols and fatty acids is preferable for removal of cis-reduced coenzyme Q₁₀, and use of a mixed solvent comprising each of hydrocarbons, alcohols, and fatty acids is particularly preferable. Use of a mixed solvent comprising hexane, 2-propanol and acetic acid is most preferable.

When used as a mixed solvent, the solvent ratio of the mixed solvent is not particularly limited and can be appropriately set in consideration of the separability between the carrier, e.g., silica gel, to be used, reduced coenzyme Q₁₀ and impurities to be removed. The ratio of the mixed solvent is not particularly limited. For example, when the above-mentioned mixed solvent of hexane, 2-propanol and acetic acid is used, the ratio of 2-propanol in the whole solvent is not more than 5% (e.g., not more than 4 wt %, not more than 3 wt %, not more than 2 wt %, or not more than 1 wt %), more preferably not more than 3%, particularly preferably not more than 1%, in the volume ratio, and the ratio of acetic acid in the whole solvent is preferably not more than 5% (e.g., not more than 4 wt %, not more than 3 wt %, not more than 2 wt %, or not more than 1 wt %), more preferably not more than 3% (e.g., not more than 2 wt %, or not more than 1 wt %), particularly preferably not more than 1%, in the volume ratio.

For removal of ubichromenol and/or reduced coenzyme Q₁₁, use of a mixed solvent comprising at least two selected from the group consisting of hydrocarbons, fatty acid esters and ethers is preferable, and use of a mixed solvent comprising hydrocarbons and alcohols is particularly preferable. Particularly, use of any one of hexane, methanol and ethanol is preferable, and most preferred is a mixed solvent comprising hexane and methanol or a mixed solvent comprising methanol and ethanol.

When used as a mixed solvent, the solvent ratio of the mixed solvent can be appropriately set in consideration of the carrier, e.g., silica gel, to be used, separability between reduced coenzyme Q₁₀ and impurities to be removed. The ratio of the mixed solvent of hexane and methanol is not particularly limited. For example, when the above-mentioned mixed solvent of hexane and methanol is used, and the ratio of methanol in the whole solvent is preferably not less than 80%, more preferably not less than 85%, particularly preferably not less than 90%, in the volume ratio. When the above-mentioned mixed solvent of methanol and ethanol is used, the ratio of methanol in the whole solvent is preferably not less than 40%, more preferably not less than 45%, particularly preferably not less than 50%, in a volume ratio. An embodiment wherein two or more reduced coenzyme Q₁₀ analogs are removed by a single operation of chromatography is also encompassed in the present invention.

Another embodiment of the production method of the crystal or composition of the present invention comprises purifying oxidized coenzyme Q₁₀ containing at least one impurity selected from the group consisting of cis-oxidized coenzyme Q₁₀, oxidized coenzyme Q₁₁, and ubichromenol by chromatography, and converting the purified oxidized coenzyme Q₁₀ to reduced coenzyme Q₁₀. To be specific, the production method of a reduced coenzyme Q₁₀ crystal or reduced coenzyme Q₁₀-containing composition comprises subjecting oxidized coenzyme Q₁₀ containing at least one impurity selected from the group consisting of cis-oxidized coenzyme Q₁₀, oxidized coenzyme Q₁₁ and ubichromenol (including a mixture of oxidized coenzyme Q₁₀ and reduced coenzyme Q₁₀) to chromatography to reduce the content of the at least one impurity, which is followed by reduction to give high quality reduced coenzyme Q₁₀. When the impurity is cis-oxidized coenzyme Q₁₀, the reduced coenzyme Q₁₀ crystal or reduced coenzyme Q₁₀-containing composition comprises not more than 1 wt %, relative to the reduced coenzyme Q₁₀, of cis-reduced coenzyme Q₁₀. When the impurity is oxidized coenzyme Q₁₁, the reduced coenzyme Q₁₀ crystal or reduced coenzyme Q₁₀-containing composition comprises not more than 1 wt %, relative to the reduced coenzyme Q₁₀, of reduced coenzyme Q₁₁. When the impurity is ubichromenol, the reduced coenzyme Q₁₀ crystal or reduced coenzyme Q₁₀-containing composition comprises not more than 1 wt %, relative to the reduced coenzyme Q₁₀, of ubichromenol.

Since the operation of purifying oxidized coenzyme Q₁₀, or oxidized coenzyme Q₁₀ and reduced coenzyme Q₁₀ by chromatography, and converting the purified oxidized coenzyme Q₁₀ to reduced coenzyme Q₁₀ does not require strict oxidation protection conditions during the chromatography step, more convenient and economical production of high quality reduced coenzyme Q₁₀ can be afforded. The carriers that can be used for the chromatography in this case are the same as those mentioned above.

As the developing solvent, the aforementioned developing solvents can be used. For removal of cis-oxidized coenzyme Q₁₀, use of a mixed solvent comprising at least two selected the group consisting of hydrocarbons, alcohols, fatty acid esters, and fatty acids is preferable, and use of a combination of hydrocarbons and alcohols or hydrocarbons and fatty acid ester is particularly preferable.

Specifically, a mixed solvent comprising two or more selected from the group consisting of hexane, 2-propanol, and ethyl acetate is preferable, and a mixed solvent comprising hexane and 2-propanol or a mixed solvent comprising hexane and ethyl acetate is more preferable. The solvent ratio of the mixed solvent is not particularly limited and can be appropriately set in consideration of the silica gel to be used and the separability between reduced coenzyme Q₁₀ and impurities to be removed. The ratio of the mixed solvent is not particularly limited. For example, when the above-mentioned mixed solvent of hexane and 2-propanol is used, the ratio of 2-propanol in the whole solvent is preferably not more than 5% (e.g., not more than 4 wt %, not more than 3 wt %, not more than 2 wt %, or not more than 1 wt %), more preferably not more than 3%, particularly preferably not more than 1%, in the volume ratio. In addition, when the above-mentioned mixed solvent of hexane and ethyl acetate is used, the ratio of ethyl acetate in the whole solvent is preferably not more than 5% (e.g., not more than 4 wt %, not more than 3 wt %, not more than 2 wt %, or not more than 1 wt %), more preferably not more than 3%, and particularly preferably not more than 1%.

For removal of ubichromenol and/or oxidized coenzyme Q₁₁, use of a mixed solvent comprising at least two selected from the group consisting of hydrocarbons, fatty acid esters, and ethers is preferable, and use of a mixed solvent comprising hydrocarbons and alcohols is more preferable. Specifically, use of any one of hexane, methanol, and ethanol is preferable, and use of a mixed solvent comprising hexane and methanol, or methanol and ethanol is most preferable. The solvent ratio of the mixed solvent is not particularly limited and can be appropriately set in consideration of the carrier, e.g., silica gel, to be used and the separability between reduced coenzyme Q₁₀ and impurities to be removed. For example, when the above-mentioned mixed solvent of hexane and methanol is used, the ratio of methanol in the whole solvent is preferably not less than 80%, more preferably not less than 85%, and particularly preferably not less than 90%.

When the above-mentioned mixed solvent of methanol and ethanol is used, the ratio of methanol in the whole solvent is preferably not less than 40%, more preferably not less than 45%, and particularly preferably not less than 50%, in the volume ratio.

The oxidized coenzyme Q₁₀, or a mixture of oxidized coenzyme Q₁₀ and reduced coenzyme Q₁₀, obtained by chromatography can be converted to high quality reduced coenzyme Q₁₀, for example, by reduction according to the method described in WO 03/06408.

The thus-obtained reduced coenzyme Q₁₀ can be processed as it is into high quality reduced coenzyme Q₁₀ by concentration, drying and the like, or crystallized according to the method described in WO 03/06408, WO 03/06409 and the like to give a substantially pure reduced coenzyme Q₁₀ crystal with a low reduced coenzyme Q₁₀ analog content. From the aspects of easy handing and higher quality, reduced coenzyme Q₁₀ crystal is more preferable.

To exert the effect of the invention to the maximum extent, for example, the method of the present invention is preferably performed and the composition of the present invention is preferably prepared and/or preserved under a deoxygenation atmosphere such as inert gas atmosphere (e.g., nitrogen atmosphere etc.) and the like.

The above-mentioned processing and preservation after processing are also preferably performed under the above-mentioned deoxygenation atmosphere such as inert gas atmosphere and the like.

According to the present invention, reduced coenzyme Q₁₀ analog, which is generally difficult to remove, can be conveniently removed, and reduced coenzyme Q₁₀ having high quality can be economically obtained with good workability.

### EXAMPLES

The present invention is explained in more detail in the following by referring to Examples, which are not to be construed as limitative. The purity of reduced coenzyme Q₁₀, the content of reduced coenzyme Q₁₀ analogs and the like in the Examples do not define the limit values of the purity in the present invention or the upper limit values thereof.

### Example 1

Reduced coenzyme Q₁₀ (5 g) containing 1.2% ubichromenol was dissolved in hexane (10 g). Reduced coenzyme Q₁₀ was adsorbed onto silica gel modified with an octadecyl group in a column, and developed with n-hexane/methanol (3/17) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.3% of ubichromenol.

### Example 2

Reduced coenzyme Q₁₀ (5 g) containing 2.1% cis-reduced coenzyme Q₁₀ was dissolved in hexane (10 g). Reduced coenzyme Q₁₀ was adsorbed onto silica gel packed in a column, and developed with n-hexane/isopropanol/acetic acid (95/0.5/0.5) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.6% of cis-reduced coenzyme Q₁₀.

### Example 3

Reduced coenzyme Q₁₀ (5 g) containing 1.2% reduced coenzyme Q₁₁ was dissolved in hexane (10 g). Reduced coenzyme Q₁₀ was adsorbed onto silica gel modified with an octadecyl group in a column, and developed with ethanol/methanol (4/3) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.3% of reduced coenzyme Q₁₁.

### Example 4

Reduced coenzyme Q₁₀ (5 g) containing 1.2% ubichromenol and 1.2% reduced coenzyme Q₁₁ was dissolved in hexane (10 g). Reduced coenzyme Q₁₀ was adsorbed onto silica gel modified with an octadecyl group in a column, and developed with hexane/methanol (1/9) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.3% of ubichromenol and 0.3% of reduced coenzyme Q₁₁.

### Example 5

Reduced coenzyme Q₁₀ (5 g) containing 1.2% ubichromenol was dissolved in hexane (10 g). Reduced coenzyme Q₁₀ was adsorbed onto silica gel modified with an octyl group in a column, and developed with n-hexane/methanol (1/9) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.8% of ubichromenol.

### Example 6

Reduced coenzyme Q₁₀ (5 g) containing 1.4% ubichromenol was dissolved in hexane (10 g). Reduced coenzyme Q₁₀ was adsorbed onto silica gel modified with an octadecyl group in a column, and developed with n-hexane/methanol (1/19) solution to give a fraction containing reduced coenzyme Q₁₀. All the solvents used were free of oxygen by repetition of reduced pressure and released pressure with nitrogen, and all the operations were performed under a nitrogen atmosphere. The solution was concentrated and dried to give reduced coenzyme Q₁₀ as crystals. The reduced coenzyme Q₁₀ contained 0.2% of ubichromenol.

### Example 7

Oxidized coenzyme Q₁₀ (5 g) containing 1.2% ubichromenol was dissolved in hexane (10 g). Oxidized coenzyme Q₁₀ was adsorbed onto silica gel modified with an octadecyl group in a column, and developed with n-hexane/methanol (1/9) solution to give a fraction containing oxidized coenzyme Q₁₀. The fraction was concentrated to give a solution (50 g) containing 4.8 g of oxidized coenzyme Q₁₀. The oxidized coenzyme Q₁₀ contained 0.4% of ubichromenol. 10% Aqueous sodium hydrosulfite solution (50 g) was added thereto and a reduction reaction was performed under a nitrogen atmosphere. After 2 hr, the aqueous phase was removed from the reaction mixture and the hexane phase was washed with deaerated saturated saline (50 g). All the operations from the reduction reaction to washing of the hexane phase with water were performed under a nitrogen atmosphere. The hexane phase was concentrated and dried to give reduced coenzyme Q₁₀ as crystals. The reduced coenzyme Q₁₀ contained 0.4% of ubichromenol.

### Comparative Example 1

Removal of Reduced Coenzyme Q₁₁ by Crystal Precipitation

Reduced coenzyme Q₁₀ (5 g) containing 1.2% reduced coenzyme Q₁₁ was dissolved in ethanol (83 g). While maintaining the same temperature, water (5 g) was added. The ethanol solution was cooled to 2° C. at a cooling rate of 10° C./hr with stirring to give a white slurry. The obtained slurry was filtered under reduced pressure, the wet crystals were washed with cold ethanol, cold water and cold ethanol in this order (temperature of cold solvent used for washing, 2° C.) and dried under reduced pressure (20-40° C., 1-30 mmHg) to give white dry crystals (4.85 g). All the operations mentioned above were performed under a nitrogen atmosphere. The obtained reduced coenzyme Q₁₀ contained 1.2% of reduced coenzyme Q₁₁.

### Comparative Example 2

Removal of Ubichromenol by Crystal Precipitation

Reduced coenzyme Q₁₀ (5 g) containing 1.4% ubichromenol was dissolved in ethanol (83 g) and dissolved at 50° C. While maintaining the same temperature, water (5 g) was added. The ethanol solution was cooled to 2° C. at a cooling rate of 10° C./hr with stirring to give a white slurry. The obtained slurry was filtered under reduced pressure, the wet crystals were washed with cold ethanol, cold water and cold ethanol in this order (temperature of cold solvent used for washing, 2° C.) and dried under reduced pressure (20-40° C., 1-30 mmHg) to give white dry crystals (4.85 g). All the operations mentioned above were performed under a nitrogen atmosphere. The obtained reduced coenzyme Q₁₀ contained 1.1% of ubichromenol.

While some of the embodiments of the present invention have been described in detail in the above, those of ordinary skill in the art can enter various modifications and changes to the particular embodiments shown without substantially departing from the novel teaching and advantages of the present invention. Such modifications and changes are encompassed in the spirit and scope of the present invention as set forth in the appended claims.

This application is based on application No. 2006-126900 filed in Japan, the contents of which are incorporated hereinto by reference.

1. A substantially pure reduced coenzyme Q₁₀ crystal wherein the content of at least one reduced coenzyme Q₁₀ analog selected from the group consisting of cis-reduced coenzyme Q₁₀, reduced coenzyme Q₁₁, and ubichromenol is not more than 1 wt %.

2. The reduced coenzyme Q₁₀ crystal of claim 1, wherein the crystal comprises not more than 1 wt % of cis-reduced coenzyme Q₁₀.

3. The reduced coenzyme Q₁₀ crystal of claim 1, wherein the crystal comprises not more than 1 wt % of reduced coenzyme Q₁₁.

4. The reduced coenzyme Q₁₀ crystal of claim 1, wherein the crystal comprises not more than 1 wt % of ubichromenol.

5. The reduced coenzyme Q₁₀ crystal of claim 1, wherein the crystal comprises not more than 3 wt % of cis-reduced coenzyme Q₁₀, reduced coenzyme Q₁₁, and ubichromenol.

6. The reduced coenzyme Q₁₀ crystal of claim 1, wherein the crystal comprises not more than 1 wt % of each of cis-reduced coenzyme Q₁₀, reduced coenzyme Q₁₁, and ubichromenol.

7. A reduced coenzyme Q₁₀-containing composition comprising reduced coenzyme Q₁₀, and not more than 1 wt %, relative to the reduced coenzyme Q₁₀, of a reduced coenzyme Q₁₀ analog selected from the group consisting of cis-reduced coenzyme Q₁₀, reduced coenzyme Q₁₁, and ubichromenol.

8. The reduced coenzyme Q₁₀-containing composition of claim 7, wherein the composition comprises not more than 1 wt % of cis-reduced coenzyme Q₁₀ relative to the reduced coenzyme Q₁₀.

9. The reduced coenzyme Q10-containing composition of claim 7, wherein the composition comprises not more than 1 wt % of reduced coenzyme Q₁₁ relative to the reduced coenzyme Q₁₀.

10. The reduced coenzyme Q₁₀-containing composition of claim 7, wherein the composition comprises not more than 1 wt % of ubichromenol relative to the reduced coenzyme Q₁₀.

11. The reduced coenzyme Q₁₀-containing composition of claim 7, wherein the composition comprises not more than 3 wt % of cis-reduced coenzyme Q₁₀, reduced coenzyme Q₁₁, and ubichromenol relative to the reduced coenzyme Q₁₀.

12. The reduced coenzyme Q₁₀ crystal of claim 7, wherein the composition comprises not more than 1 wt % of each of cis-reduced coenzyme Q₁₀, reduced coenzyme Q₁₁, and ubichromenol relative to the reduced coenzyme Q₁₀.

13. A method of producing a reduced coenzyme Q₁₀ crystal or reduced coenzyme Q₁₀-containing composition, which comprises
providing a solution comprising reduced coenzyme Q₁₀, at least one reduced coenzyme Q₁₀ analog selected from the group consisting of cis-reduced coenzyme Q₁₀, reduced coenzyme Q₁₁, and ubichromenol, and a solvent, and
subjecting the solution to chromatography to reduce the concentration of the at least one reduced coenzyme Q₁₀ analog to not more than 1 wt % relative to the reduced coenzyme Q₁₀.

14. The method of claim 13, wherein the chromatography is column chromatography.

15. The method of claim 13, wherein a carrier of the chromatography is silica gel or chemically-modified silica gel.

16. The method of claim 15, wherein the chemically-modified silica gel is silica gel modified by an octadecyl group.

17. The method of claim 13, wherein the chromatography is performed under an environment where reduced coenzyme Q₁₀ is protected from oxidation.

18. The method of claim 13, wherein subjecting the solution to chromatography comprises contacting a carrier with the solution and then contacting the carrier with a developing solvent.

19. The method of claim 18, wherein the developing solvent is a mixed solvent of hexane, 2-propanol, and acetic acid, and the reduced coenzyme Q₁₀ analog is cis-reduced coenzyme Q₁₀.

20. The method of claim 18, wherein the developing solvent is selected from the group consisting of hexane, methanol, ethanol and mixed solvents thereof, and the reduced coenzyme Q₁₀ analog is one or both of reduced coenzyme Q₁₁ and ubichromenol.

21. A method of producing a reduced coenzyme Q₁₀ crystal or reduced coenzyme Q₁₀-containing composition, which comprises
providing a solution comprising (a) oxidized coenzyme Q₁₀, (b) at least one impurity selected from the group consisting of cis-oxidized coenzyme Q₁₀, oxidized coenzyme Q₁₁, and ubichromenol, and (c) a solvent,
subjecting the solution to chromatography to reduce the content of the at least one impurity, thereby producing a purified oxidized coenzyme Q₁₀, and
converting the purified oxidized coenzyme Q₁₀ to a reduced coenzyme Q₁₀ that contains not more than 1 wt %, relative to the reduced coenzyme Q₁₀, of (a) cis-reduced coenzyme Q₁₀ if the impurity is cis-oxidized coenzyme Q₁₀, (b) reduced coenzyme Q₁₁ if the impurity is oxidized coenzyme Q₁₁, or (c) ubichromenol if the impurity is ubichromenol.

22. The method of claim 21, wherein subjecting the solution to chromatography comprises contacting a carrier with the solution and then contacting the carrier with a developing solvent.

23. The method of claim 22, wherein the developing solvent is a mixed solvent comprising at least two of the group consisting of hexane, 2-propanol, and ethyl acetate, and the impurity is cis-oxidized coenzyme Q₁₀.

24. The method of claim 22, wherein the developing solvent is selected from the group consisting of hexane, methanol, ethanol and mixed solvents thereof, and the impurity is one or both of oxidized coenzyme Q₁₁ and ubichromenol.
